(19) United States
(12) Patent Application Publication
(10) Pub. No.: US 2026/0174344 A1
(43) Pub. Date: Jun. 25, 2026

(54) WEARABLE DEVICES FOR FETUS PHYSIOLOGICAL CONDITION DETERMINATION

(71) Applicant: OXITONE MEDICAL LTD., Kfar Saba (IL)

(72) Inventors: Reuven GLADSHTEIN, Kfar Saba (IL); Leon EISEN, Kfar Saba (IL); Bar EPSTEIN, Kfar Saba (IL); Ofer HARPAK, Kfar Saba (IL); Meital GOLTS, Kfar Saba (IL)

(21) Appl. No.: 19/128,970

(22) PCT Filed: Nov. 10, 2023
(86) PCT No.: PCT/IB2023/061391

§ 371 Date: May 9, 2025

Related U.S. Application Data
(60) Provisional application No. 63/424,290, filed on Nov. 10, 2022.

(51) Int. Cl. A61B 5/024; A61B 5/00; A61B 5/022; G16H 40/67; G16H 50/20

(52) U.S. Cl. CPC A61B 5/02411; A61B 5/02233; A61B 5/02405; A61B 5/02427; A61B 5/681; A61B 5/6823; A61B 5/7246; A61B 5/725; A61B 5/7405; A61B 5/746; G16H 40/67; G16H 50/20

(57) ABSTRACT

In embodiments of the present disclosure, a device and method for determining a heart rate of a fetus and a heart rate variability of the pregnant woman and the fetus are contemplated. The method includes generating a spectral transform from a waveform representative of a hemodynamic parameter specific to the individual, determining candidate remnant signals included in an area independent of exclusion areas of the spectral transform, identifying a candidate remnant signal from the candidate remnant signals, comparing the candidate remnant signal with a target signal representative of oxygen levels present in blood stream of the individual, the target signal is derived from the hemodynamic parameter, and determining that the candidate remnant signal is associated with a heart rate of the fetus that is disposed in the individual upon identifying an inverse correlation between the candidate remnant signal and the target signal of the individual.

### CROSS REFERENCE TO RELATED APPLICATIONS

The present application claims priority to U.S. Provisional Patent Application No. 63/424,290 entitled “Wearable Devices for Fetus Physiological Condition Determination” filed Nov. 10, 2022, which is hereby incorporated by reference in its entirety.

### TECHNICAL FIELD

Embodiments of the present invention relate to a device that can be worn on a wrist or disposed on a chest area of an individual, in addition to various methods and applications associated therewith.

### BACKGROUND

This section is intended to introduce various aspects that may be related to embodiments of the present invention, which are described and/or claimed below. This discussion is believed to be helpful in providing background information to facilitate a better understanding of the various aspects of embodiments of the present invention. Accordingly, it should be understood that these statements are to be read in this light, and not as admissions of prior art.

A wide variety of devices have been developed for monitoring physiological characteristics of a patient. Such devices provide patients, doctors, and other healthcare personnel with the information they need to secure the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood characteristics, such as the arterial blood oxygen saturation of hemoglobin (SPO2), and the rate of blood pulsations corresponding to each heartbeat of a patient. In fact, the “pulse” in pulse oximetry refers to the time varying amount of arterial blood at the measurement site during each cardiac cycle. Those skilled in the art will appreciate the pulse oximetry techniques used for obtaining the above physiological parameters which may also be termed photoplethysmography or, in short, PPG.

In the field of medicine, it is also desirable to regularly monitor the physical condition of a pregnant woman and the fetus that she is carrying. In particular, monitoring physiological parameters of the woman and determining and/or deriving one or more physiological characteristics of the fetus based on these parameters, namely hemodynamic parameters, may enable the determination of any sudden changes in the health of the woman and/or the condition of the fetus. Several such devices are currently available, but are based on Doppler and Cardiotocography (“CTG”) technology. As such, these devices are required to be placed directly on the abdomen of the mother for detecting a fetus heart rate. As such, these devices do not enable real-time monitoring of the health of the mother and that of the fetus for a prolonged period of time.

The following patent disclosures by the applicant are hereby incorporated by reference herein in their entireties: U.S. Pat. No. 9,314,197, titled “Wearable pulse oximetry device,” U.S. Pat. Nos. 8,868,149 and 9,149,216, each titled “Photoplethysmography device and method,” PCT Application Number PCT/IB2017/058022 titled “Wrist-Sensor Pulse Oximetry Device and Method,” and US Patent Application Publication Number 2020/0015,723 titled “Wrist-Sensor Pulse Oximetry Device And Method.”

### SUMMARY

In embodiments of the present disclosure, devices and methods that address and overcome various deficiencies are contemplated. In particular, the device and method for determining a heart rate of a fetus and a heart rate variability of the pregnant woman and that of the fetus are contemplated. The system may include a device that utilizes photoplethysmography (“PPG”) optical technology (e.g., a PPG sensor) to determine various physiological parameters of the pregnant woman and utilizes at least one of these parameters to determine a physiological parameter of a fetus carried by the woman. While such a device may be positioned on the wrist of the pregnant woman, any location on the body from where a pulse may be determined is contemplated. In embodiments, a device in the form of a chest patch may be disposed on the chest area of a pregnant woman and may be utilized to obtain one or more hemodynamic parameters specific to the woman, which in turn is utilized to determine, e.g., a heart rate of a fetus that she may be carrying.

In one embodiment, a method of determining at least a physiological parameter specific to a fetus disposed in an individual comprises generating a spectral transform from a waveform representative of a hemodynamic parameter specific to the individual, the spectral transform including a plurality of harmonics within a sampling period, the plurality of harmonics representative of the hemodynamic parameter, determining a plurality of candidate remnant signals included in an area independent of exclusion areas of the spectral transform, identifying a candidate remnant signal from the plurality of candidate remnant signals, comparing the candidate remnant signal with a target signal representative of oxygen levels present in blood stream of the individual, the target signal is derived from the hemodynamic parameter, and determining that the candidate remnant signal is associated with a heart rate of the fetus that is disposed in the individual upon identifying an inverse correlation between the candidate remnant signal and the target signal of the individual.

In another embodiment, a device comprises a sensor, at least one light source, a processor, a network communication interface, and memory. The processor is configured to generate a spectral transform from a waveform representative of a hemodynamic parameter specific to an individual, the spectral transform including a plurality of harmonics within a sampling period, the plurality of harmonics representative of the hemodynamic parameter, determine a plurality of candidate remnant signals included in an area independent of exclusion areas of the spectral transform, identify a candidate remnant signal from the plurality of candidate remnant signals, compare the candidate remnant signal with a target signal representative of oxygen levels present in blood stream of the individual, the target signal is derived from the hemodynamic parameter, and determine that the candidate remnant signal is associated with a heart rate of the fetus that is disposed in the individual upon identifying an inverse correlation between the candidate remnant signal and the target signal of the individual.

### BRIEF DESCRIPTION OF DRAWINGS

For a better understanding of some embodiments of the present invention and to show how the same may be carried into effect, reference will now be made, by way of example, to the accompanying drawings in which like numerals designate corresponding elements or sections throughout. In the accompanying drawings:

FIG. 1 is an illustration of a pulse oximeter;

FIG. 2 is a perspective view of a wrist wearable device in accordance with an exemplary embodiment of the present invention;

FIG. 3 depicts another embodiment and a perspective view of an example device that may be disposed on the chest of a pregnant woman;

FIGS. 4A and 4B depict an example implementations of applications of the example device of the present disclosure, according to one or more embodiments described and illustrated herein;

FIG. 5 depicts two distinct example graphical representations that include physiological data specific to an individual;

FIG. 6 depicts three distinct graphical representations that are a result of one or more processing operations performed on sampled hemodynamic waveforms associated with PPG signals, according to one or more embodiments described and illustrated herein;

FIG. 7 depicts two distinct graphical representations associated with a variability of a heart rate of a woman (e.g., a pregnant woman) generated as a result of one or more processing operations performed on sampled hemodynamic waveforms associated with PPG signals, according to one or more embodiments described and illustrated herein;

FIG. 8 depicts a work flow illustrating an example implementation of the method and device of the present disclosure, according to one or more embodiments described and illustrated herein;

FIG. 9 depicts a graphical representation of the various harmonic components of a filtered and detrended hemodynamic signal, according to one or more embodiments described and illustrated herein;

FIG. 10 depicts a graphical representation of remnant signal area and from one or more candidate signals may be utilized to potentially determine a heart rate of a fetus, according to one or more embodiments described and illustrated herein; and

FIG. 11 depicts the presence of remnant signals that may be further analyzed to determine a heart rate of a fetus, according to one or more embodiments described and illustrated herein.

### DETAILED DESCRIPTION

With specific reference now to the drawings in detail, it is to be understood that the particulars shown are by way of example and for purposes of illustrative discussion of preferred embodiments of the present invention only. The description taken in conjunction with the drawings will make apparent to those of ordinary skill in the art how the several forms and embodiments of the invention may be embodied in practice.

It is also to be understood that embodiments of the invention are not limited in their application to the details of construction and the arrangement of the components set forth in the following description or illustrated in the drawings. Embodiments of the invention may be practiced or carried out in various other ways. In addition, it is to be understood that the phraseology and terminology employed herein is for the purpose of description and should not be regarded as limiting.

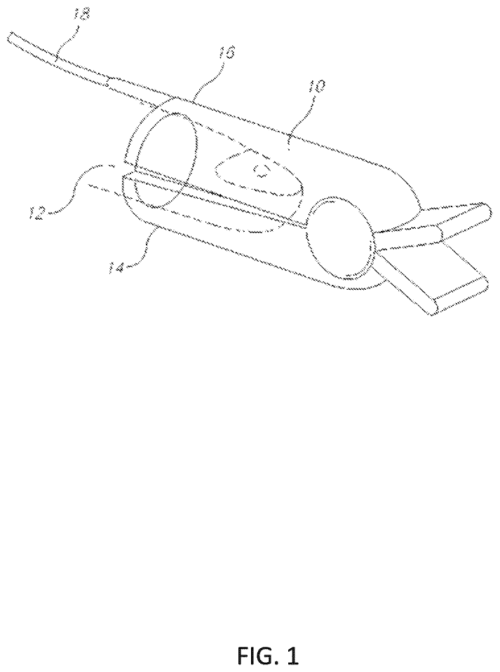

FIG. 1 illustrates a pulse oximeter. In particular, FIG. 1 illustrates a sensor 10 adapted to be placed on a finger 12 of a user, such as a patient. The sensor 10 includes a clip formed of two clip portions 14 and 16 adapted to clip and constrain the sensor 10 to finger 12 while pulse oximetry measurements are taken. Sensors of a type similar to the sensor 10 are typically coupled to cables 18 that couple the sensor 10 to monitoring systems adapted to receive and process the signals from the sensor 10. Accordingly, such a sensor, when used in continuous monitoring mode, typically requires the patient (or user) to be confined to a certain area, in close vicinity of the monitoring system, thereby limiting patient mobility. In addition, pinch pressure applied by clip portions 14 and 16 on the finger 12 of the patient may, overtime, cause the patient to feel discomfort, thereby requiring the patient to remove the sensor 10. As a result, such sensors are not suitable for prolonged and continuous pulse oximetry measurements.

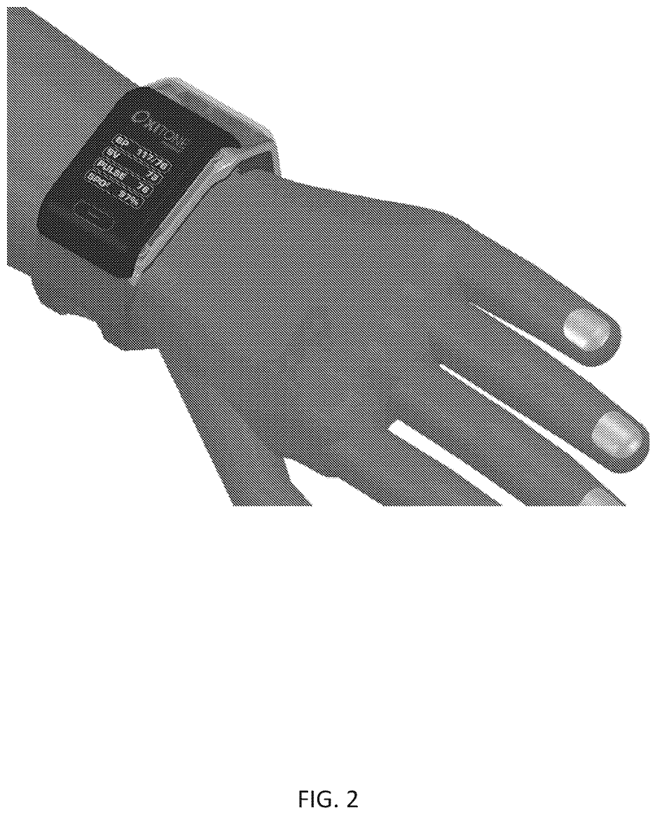

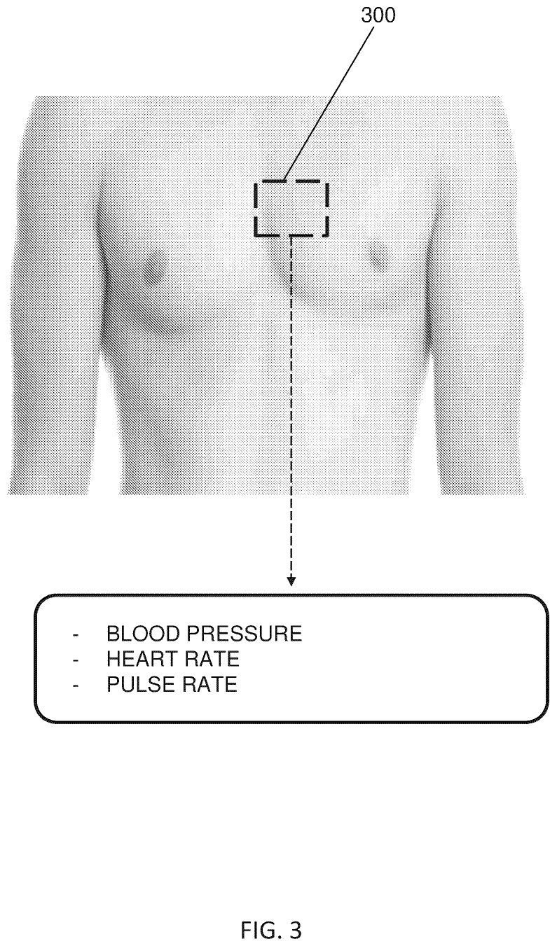

The devices illustrated in FIGS. 2 and 3 address and overcome various deficiencies.

Turning now to the figures, FIG. 2 is a perspective view of a device 200 (e.g., a wrist wearable device) in accordance with an exemplary embodiment of the present invention. Device 200 may be a wrist-type oximeter device adapted to be worn on a wrist of a user, as further shown in FIG. 2. In some embodiments, device 200 is adapted to obtain data including, for example, pulse data, oxygen saturation (SPO2) data, and/or other data from a user while the user wears device 200 on the wrist. Hence, a user can wear the device 200 in a manner similar to that of wearing a watch, a wrist band or any article of clothing, ornament, or garment adapted to be worn on the wrist of the user, and so forth. In this manner, a user can wear device 200 while performing any routine and ordinary operation the user would otherwise perform in everyday life, such as walking, running, cycling and so forth. In accordance with embodiments of the present disclosure, device 200 can be conveniently worn at any time or place by those users required to or wishing to obtain, for example, pulse oximetry and pulse rate data without being attached to elaborate monitoring device or being confined to certain monitoring areas. Thus, the device 200 is a self-contained, self-powered device adapted to obtain, analyze and process, for example, various light electromagnetic signals from which pulse oximetry data is ultimately obtained. Device 200 may further include wired or wireless interfaces whereby the device 200 can communicate and/or relay data signals to external and/or remote devices. Hence, in some embodiments, device 200 can collect and provide the oximetry data to any remote users, institutions such as hospitals or clinics, or anyone who requires or has interest in such pulse oximetry data of the user.

As illustrated in FIG. 2, device 200 may include a display 202 that displays, for example, data measured by device 200. Such data may include pulse rate data (e.g., “PULSE 76”), and data regarding the wearer's blood oxygen saturation of hemoglobin (e.g., “SPO² 97%”). In some embodiments, display 202 may be an LED display, such as, for example, an organic light-emitting diode (“OLED”) display, liquid crystal display (“LCD”), or any other suitable display. In some embodiments, device 200 may include one or more physical buttons or user input interfaces (e.g., alphanumerical buttons or user interface where by the user can enter any combination of numbers and/or letters as desired or needed while the device is in use). Alternatively or additionally, in some embodiments, one or more buttons or user interface inputs may be placed at any side, or sides, of device 200 or any other area of device 200 that is accessible to the user. In some embodiments, device 200 may alternatively or additionally measure and/or display other data, including, for example, data regarding one or more vital signs, data regarding one or more blood analytes, blood pressure data (e.g., “BP 117/76”), and/or data regarding stroke volume (e.g., “SV 73”).

FIG. 3 depicts another embodiment and a perspective view of an example device 300 that may be disposed on the chest of an individual, e.g., a pregnant woman. In embodiments, the example device 300 may be a patch that is worn in a chest area of a woman serves to monitor various physiological parameters specific to the woman, e.g., data related to hemodynamic parameters such as a heart rate, pulse rate, and blood pressure may be obtained in real time. Further, in embodiments, various positions of a pregnant woman during the pregnancy may be monitored and determined in real time. For example, the device 302 and/or the device 200 may be utilized to determine postures such as, e.g., sitting, standing, lying down, and so forth. Moreover, the precise position in which the woman may be lying may also be determined. Data specific to these physical postures or orientations may be correlated with various hemodynamic parameters such as, e.g., heart rate, pulse rate, blood pressure, and so forth. The correlation may be utilized to generate recommendations of particular positions that are suitable for the pregnant mother. In embodiments, these recommendations may be displayed on a display of the device 200. In some embodiments, these recommendations may be wirelessly transmitted by the example device 300 to, e.g., a smartphone of the pregnant woman, and output on the display of the smartphone. Other such examples are also contemplated. In embodiments, data specific to blood pressure, heart rate, pulse rate, and so forth, may also be transmitted by the example device 300 to a smartphone of the pregnant woman. The transmitted hemodynamic data may be output on the smartphone of the pregnant woman.

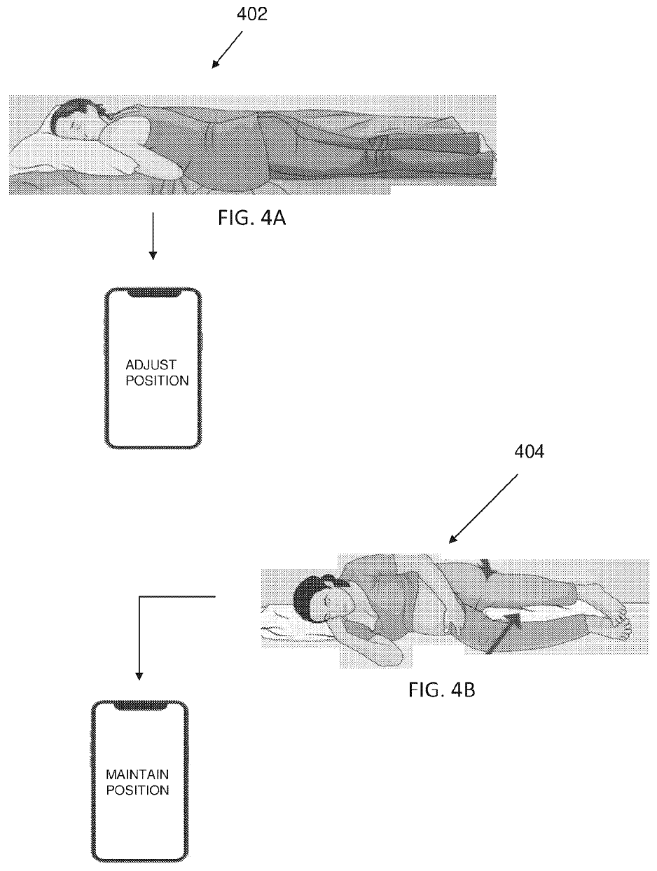

FIG. 4A depicts an example implementation of an application of the example device 300 of the present disclosure, according to one or more embodiments described and illustrated herein. FIG. 4A illustrates a pregnant woman that is lying down in a first posture 402, which is an unadvisable position. In particular, the first posture 402 is such that the pregnant woman appears to be pressing her belly against the surface of the mattress, thereby potentially squeezing and harming the health of the fetus. As such, the device 200 or the example device 300 may determine, in real time, the orientation of the pregnant woman and generate an audio alarm and/or an audiovisual alarm. In embodiments, the audio alarm may be a beeping sound that informs the pregnant woman that she is positioned in an unadvisable physical orientation. In embodiments, the audiovisual alarm may be transmitted to a smartphone of the pregnant woman and output on the screen of the smartphone. For example, the audiovisual alarm may be output by a smartphone of the pregnant woman as a result of transmission from the example device 300. In embodiments, the audiovisual alarm may be a text message such as, e.g., “ADJUST POSITION” combined with a beeping sound that is output by the example device 300. In embodiments, the smartphone may also vibrate.

FIG. 4B depicts an example implementation of an application of the example device 300 of the present disclosure, according to one or more embodiments described and illustrated herein. FIG. 4B depicts a second posture 404 or orientation that may be detected by the device 200 or the example device 300. The second posture 404 is one that may be determined to be an advisable position because, as illustrated, the pregnant woman appears to have placed a pillow between her legs and is leaning on her right side such that the side of her stomach appears to be disposed on the mattress. Such a position does not appear to put any pressure on the fetus. The example device 300 may generate an audio alarm in the form of a sound that is different from the sound described above, indicating that the current orientation of the pregnant woman is safe and advisable, and as such, should be maintained. Similarly, the example device 300 may transmit an audiovisual recommendation that may be output on, e.g., the smartphone of the pregnant woman. In embodiments, the audiovisual recommendation may be a text message such as “MAINTAIN POSITION” or “MAINTAIN CURRENT POSITION” combined with an audio output such as a beeping sound with a pattern or frequency that varies from the beeping sound described above with respect to FIG. 4A.

In embodiments, it is noted that while the device 200 may be a wrist wearable device and the example device 300 may be patch wearable on a chest of an individual, other designs, shapes, dimensions, and configurations of these devices are also contemplated. Further, each of the device 200 and the device 300 may include at least one light source, at least one sensor, a processor, memory, a communication interface, and a battery. Further, each device is communicatively coupled with one or more external devices, e.g., a server, a smartphone, a laptop, and so forth, and the processor is operable to execute a software application that enables for the determination of various physiological and hemodynamic parameters that are utilized to determine at least a heart rate of a fetus, in addition to various physiological conditions of the pregnant woman. In embodiments, each of these devices may also include an accelerometer that serves to enable the determination of a position, movement, or orientation of an individual.

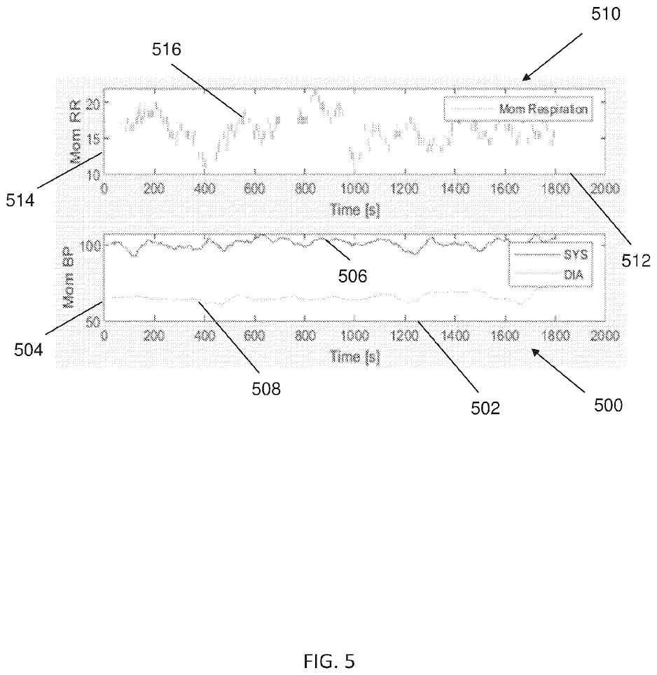

FIG. 5 depicts two distinct example graphical representations that include physiological data specific to an individual. The data may be collected using a PPG sensor based device, e.g., device 200 and/or example device 300. In particular, a first example graphical representation 500 depicts time values on an example x-axis 502 and blood pressure values on an example y-axis 504. In particular, the first example graphical representation 500 depicts a systolic blood pressure curve 506 (indicated in purple) and a diastolic blood pressure curve 508 (indicated in blue) over a particular time frame, e.g., a time frame ranging from 0 to 1800 seconds. A second example graphical representation depicts time values on an example x-axis 512 and respiration rate on an example y-axis 514. In particular, the variation in the respiration rate of, e.g., a pregnant woman, is illustrated using respiration curve 516 (indicated in green).

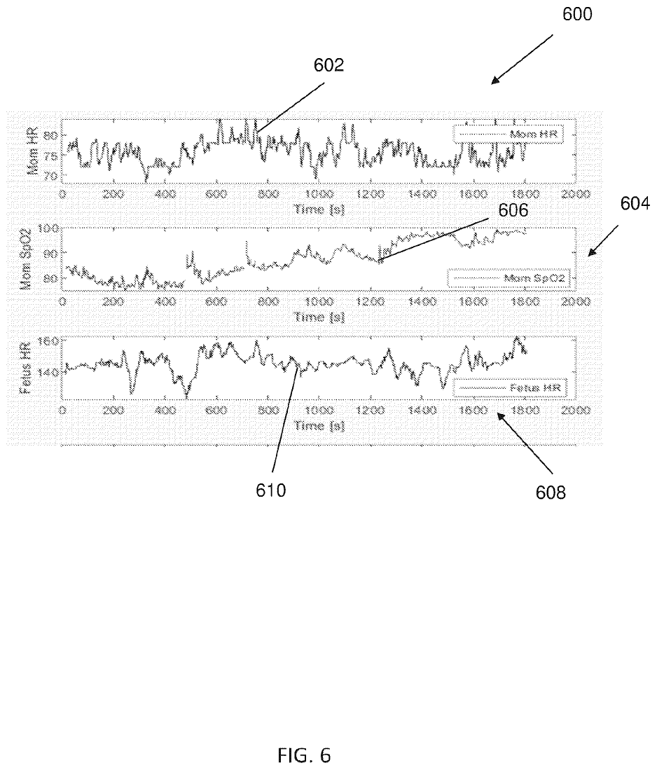

FIG. 6 depicts three distinct graphical representations that are a result of one or more processing operations performed on sampled hemodynamic waveforms associated with PPG signals, according to one or more embodiments described and illustrated herein. In particular, a third example graphical representation 600 depicts a heart rate curve 602 (indicated in blue), an oxygen level curve 606 (indicated in red), and a fetus heart rate curve 610 (indicated in blue).

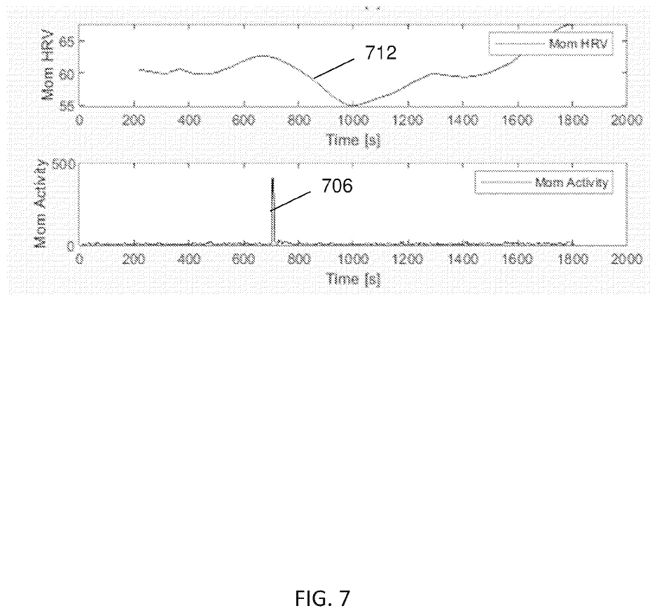

FIG. 7 depicts two distinct graphical representations associated with a variability of a heart rate of a woman (e.g., a pregnant woman) generated as a result of one or more processing operations performed on sampled hemodynamic waveforms associated with PPG signals, according to one or more embodiments described and illustrated herein. As illustrated, the example heart rate variability curve 712 ranges from 200 seconds to 1800 seconds with a lowest value at a time of 1000 seconds and a highest value at a time of 1800 seconds. Additionally, as illustrated, a general activity curve 706 ranges from 0 seconds to 1800 seconds and shows a sudden spike of activity at approximately the 700 second mark.

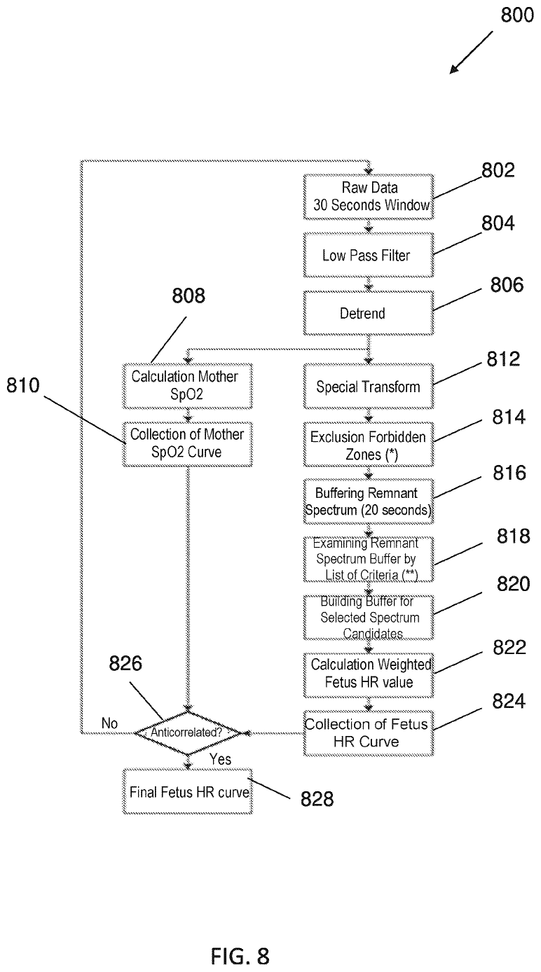

FIG. 8 depicts a work flow 800 illustrating an example implementation of the method and device of the present disclosure, according to one or more embodiments described and illustrated herein. Prior to detailing the steps included as part of the workflow 800, an overview of the method steps for determining the physiological parameters of the fetus may be instructive.

The steps for determining a physiological parameter of the fetus may be based on processing of measured hemodynamic signals. In particular, the device 200 and 300 measure hemodynamic signals in the form of pulse wave forms, which are processed in order to determine blood pressure data, respiratory rate data, heart rate data, heart variability data, and so forth. These signals may be photoplethysmographic (PPG signal based) or may be based on an ultrasonic reading or a bio-impedance reading. Thereafter, as illustrated in FIGS. 6 and 7, various physiologic parameters may be calculated from the extracted signals as part of one or more processing operations. Such physiologic parameters may be calculated in a time dimension or a frequency dimension. In embodiments, the calculation of the physiologic parameters from the signals extracted from the pregnant woman is a part of determining one or more signals that are representative of a fetus heart rate. It is noted that calculation of the physiologic parameters is associated with signals of hemodynamic waveforms of PPG nature that are directly sampled. The signals may be of an ultrasonic or electrical nature also. Further, it is noted that the signals that are graphically represented in FIGS. 6 and 7 are as a result of processing operations performed on sampled hemodynamic wave forms of PPG.

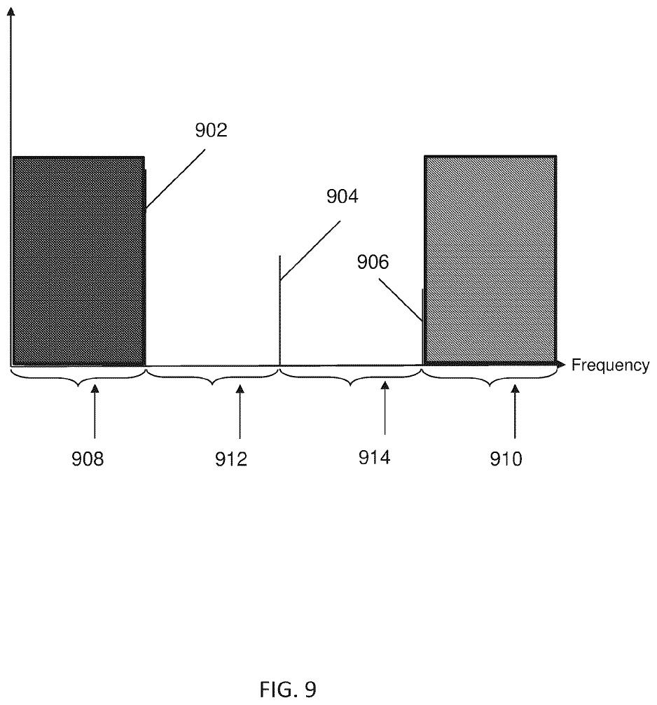

In operation and during implementation, signal components corresponding to the first harmonic, the second harmonic, and the third harmonic may be associated with physiological parameters specific to the pregnant woman, e.g, hemodynamic parameters such as heart rate, blood pressure, and so forth. Further, particular excluded spectral areas or forbidden zones (illustrated in FIGS. 9-11 and described in greater detail below) may be identified and designated. These forbidden zones are found in spectral areas where the signal frequencies are less than 1.7 Hz and more than 4 Hz. In addition, each of the first harmonic, the second harmonic, and the third harmonic, and respective thickness ranges for each of these harmonics are also included in the forbidden zones. In sum, the forbidden zones do not correspond with or do not include signals (e.g., remnant signals as described below) that may be utilized to determine an accurate and final heart rate of a fetus.

In operation and during implementation, a particular spectral component of signal (e.g., remnant signal) may be identified and/or selected for determining a fetus heart rate, e.g., by assessing whether the signal spectral component fits or satisfies a particular set of numerical properties, namely properties that are associated with common physiological parameters of fetuses. It is noted that this signal is extracted or identified from a spectral area that is independent of the exclusion areas. For example, average fetus heart rate variation levels, average margin of fetus heart rate amplitude change, and so forth, may be utilized as numerical properties that should be satisfied for associating a signal component with a fetus physiological parameter. It is noted that the comparison process will be described in further detail below.

Finally, the extracted or determined signal may be validated using at least one of the physiologic parameters, calculated from the signal specific to the pregnant woman. Methods of validation may involve correlating the dynamics of the fetus parameter with the at least one physiological parameter of the mother.

Returning to the work flow 800 illustrated in FIG. 8, it is noted that hemodynamic signals such as, e.g., pulse wave forms, may be obtained using the device 200 that is worn on a wrist of a pregnant woman or the example device 300 that is disposed on a chest of the woman, as illustrated in FIG. 3. In embodiments, the physiological data that is obtained may be stored in the memory of the device 200 and/or the memory of the device 300. In embodiments, the collected data may be transmitted to one or more devices that are external to the devices 200, 300 (e.g., one or more servers). In embodiments, the hemodynamic signal that is obtained by the device 200 or the device 300 may take the form of a blood pressure wave, which is distinctive in that it is a pulsative signal associated with one or more arteries of heart. In embodiments, this pulsative signal may be captured over various time periods or sampling windows, e.g., a 30 second sampling window, a 60 second sampling window, and so forth (block 802).

In embodiments, subsequent to capturing the hemodynamic data (e.g., represented by a blood pressure wave), one or more signal processing operations may be performed on the captured hemodynamic data. In embodiments, these signal processing operations may be performed manually or automatically and without user intervention. In particular, a low pass filter may be applied to the obtained hemodynamic data, represented by the blood pressure wave (block 804) cleaned from high frequency noises. As a result, data associated with frequencies above a particular threshold value are filtered out from the blood pressure wave and only those frequencies at or below the threshold value are retained. In this way, the application of the low pass filter removes frequency values that correspond to noise or interference.

In embodiments, an additional signal processing operation may be performed on the signal, namely a detrending operation (block 806). In embodiments, a detrending operation corresponds to the application of a function that removes a “best-fit line” from a dataset, in this case, the detrended blood pressure waveform data that is represented by the aligned blood-pressure waveform. In embodiments, the use of the detrending operation or function enables for the identification of cyclical patterns or other comparable patterns in waveforms. Moreover, the detrending operations enables for the identification of sudden increases, decreases, or comparable variations present in datasets, waveforms, and so forth.

In embodiments, subsequent to the performing of the detrending operation, a spectral transform may be generated, manually or automatically and without user intervention (block 812). The generation of the spectral transform enables for the graphical representation of the respective harmonic components of the detrended signal. It is noted that the graphical representations of the harmonic components of the detrended signal are illustrated in FIGS. 9-11 and described in greater detail below. Further, it is noted that, another output from the detrending operation may be a calculation of oxygen levels present in the blood stream of, e.g., the pregnant woman (block 808). It is noted that the oxygen level calculations may be collected (block 810) and potentially stored, e.g., in memory of the device 200 and/or the example device 300.

Subsequent to the performing of the spectral transform, a buffering operation in association with a remnant spectrum may be performed (block 816) and the remnant spectrum may be compared with a list of criteria. As described above, the remnant spectrum may be compared with a list of criteria (e.g., average fetus heart rate variation rates, average margin of fetus heart rate amplitude change, and so forth) (block 818). Thereafter, a buffer may be built for particular spectrum candidates (block 820) and a weighted fetus heart rate value may be determined (block 822). Finally, a heart rate curve specific to the fetus may be generated (block 824) and a correlation may be performed between the collection of the oxygen level calculations associated with the pregnant woman, as determined in block 810 and the heart rate curve generated in step 824. In embodiments, if there is no anti-correlation between the oxygen level calculations and the fetus HR curve, namely an inverse correlation, the process flow proceeds to block 802. If, however, it is determined that there is an anti-correlation (i.e. inverse correlation), such that the inverse correlation meets or exceeds a particular threshold, the heart rate curve determined in block 824 may be classified as an accurate and final heart rate curve of a fetus. The above is a summary of the work flow 800, while a more detailed description of at least a subset of the steps 812-824 is provided below.

FIG. 9 depicts a graphical representation of the various harmonic components of a filtered and detrended hemodynamic signal, according to one or more embodiments described and illustrated herein. In FIG. 9, a first harmonic 902, a second harmonic 904, and a third harmonic 906 (above 4 Hz) of the heart rate of the pregnant woman is shown. The first, second, and third harmonics 902, 904, and 906 and frequencies above 4 Hz and below 1.7 Hz are identified for the purposes of exclusion, which in turn enables for the identification of one or more additional candidate signals corresponding to the heart rate of the fetus.

In embodiments, as part of the workflow illustrated in FIG. 8, the frequencies corresponding to the first harmonic 902, second harmonic 904, the third harmonic 906 and frequencies above 4 Hz are identified, manually or automatically and without user intervention, as exclusion areas or “forbidden zones.” In other words, the first, second, and third harmonics, 902, 904, 906 and frequencies above 4 Hz and below 1.7 Hz are not included as or may not be classified as candidate signals for the heart rate of the fetus. It is noted that the magnitude of the forbidden zones incorporate the width of each of the harmonics, and as such, are slightly larger than the frequencies corresponding to the first, second, and third harmonics 902, 904 and 906.

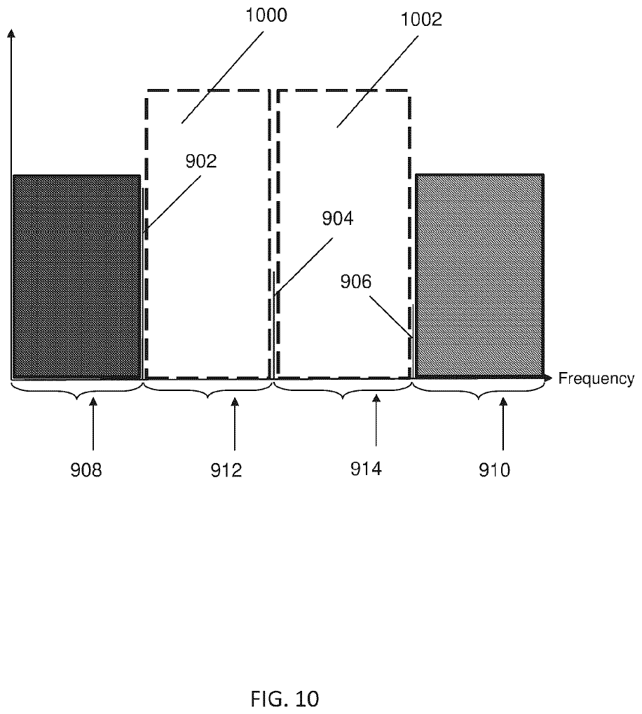

Further, in embodiments, the forbidden zones include a frequency range that is above a particular threshold frequency and a frequency range below a particular threshold frequency. For example, as illustrated in FIG. 10, all frequencies below the frequency corresponding to, e.g., less than 1.7 Hz, and all frequencies above the frequency corresponding to, e.g., 4 Hz, are included as part of the forbidden zones or zones. In other words, signals within these zones may not correspond to or may not be utilized to determine a heart rate of a fetus. All frequencies below the first harmonic 902 are indicated with a brown rectangle (representative of frequency range 908) and all frequencies above 4 Hz are indicated with a blue rectangle (representative of frequency range 910). In sum, the forbidden zones include the frequency range below the first harmonic 902, the frequency range above 4 Hz, the frequency range below 1.7 Hz and the frequency values corresponding to the first harmonic 902, the second harmonic 904, and the third harmonic 906. As such, any frequencies that fall outside the range of the identified forbidden zones may be classified as candidate signals (e.g., remnant signals) that may be utilized to determine the heart rate of the fetus. It is noted that the identification of the forbidden zones, as described above and illustrated in FIG. 9, correspond to block 814 described above.

FIG. 10 depicts a graphical representation of remnant signal area 1000 and 1002 from one or more candidate signals may be utilized to potentially determine a heart rate of a fetus, according to one or more embodiments described and illustrated herein. As illustrated, the remnant signal areas 1000 and 1002 are identified in between the first and second harmonics 902 and 904, and between the second and third harmonics 904 and 906. (below 4 Hz).

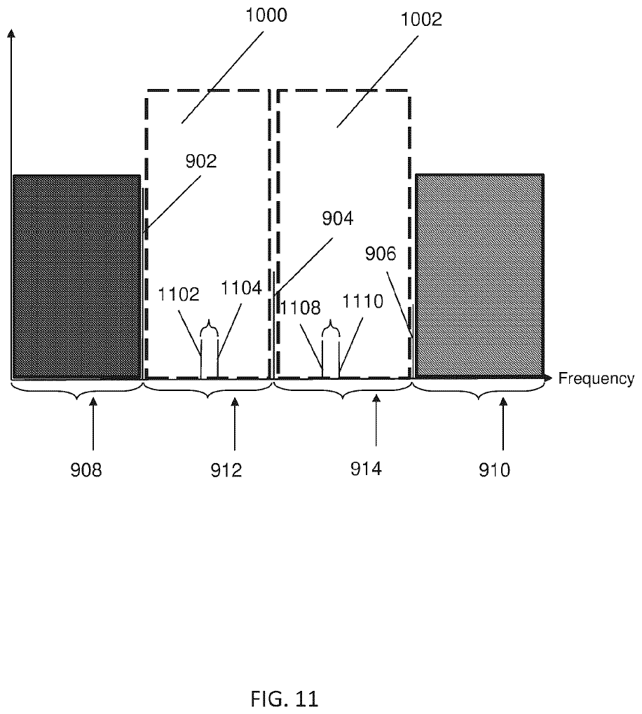

FIG. 11 depicts the presence of remnant signals that may be further analyzed to determine a heart rate of a fetus, according to one or more embodiments described and illustrated herein. In particular, as illustrated in FIG. 11, remnant signals 1102, 1104 are illustrated as being present between the first harmonic 902 and the second harmonic 904 and the remnant signals 1108, 1110 are additional remnant signals illustrated as being present between the second harmonic 904 and the third harmonic 906 (when their values are above 1.7 Hz and below 4 Hz).

As part of identifying the remnant signals 1102, 1104, 1108, and 1110 that are deemed as the appropriate remnant signals, namely remnant signals that may be utilized to determine a heart rate of a fetus, these may be observed within a 20 second window. Such an observation process is the step of buffering of the remnant spectrum (block 816). At the end of the step performed in block 816, a particular set of remnant signals within a 20 second window are identified as potentially appropriate remnants for determining a heart rate of the fetus. The steps for identifying the appropriate remnant signals are described below.

In embodiments, after a set of candidate remnant signals within a 20 second window are identified, these candidate remnant signals are correlated with or examined relative to a list of criteria, namely one or more numerical properties that characterize a standard or typical physiological behavior of a fetus (block 818). In embodiments, the numerical criteria may correspond to a particular numerical value representing an average variation in heart rate, an average variation in amplitude of a heart rate, an average change in heart rate, and so forth. If the candidate remnant signals fail to satisfy one or more of the list of numerical criteria, the candidate remnant signals are filtered out. In other words, these signals are not determined to be suitable signals from which a heart rate of a fetus may be determined. Conversely, one or more remnant signals that do satisfy the numerical criteria (e.g., associated with the average variation in heart rate, the average variation in amplitude of a heart rate, and the average change in heart rate) are identified as appropriate remnant signals from which a heart rate of the fetus may be determined (block 820). After having determined the appropriate remnant signals, weighted coefficients may be determined for the remnant signals.

In particular, in embodiments, weighted coefficients may be determined for remnant signals 1102, 1104, 1106, and 1108 such that these sets of signals may be merged to determine a single remnant signal (block 822). For example, in embodiments, weighted coefficients may correspond to weighted averages of the remnant signals 1102, 1104, 1106, and 1108, which are then applied to these signals in order to merge them to generate a single signal. For example the remnant signals 1102 and 1104 may be merged to generate a single signal and remnant signals 1106 and 1008 may be merged to generate another single signal.

After calculation of the weighted coefficients, the remnant signals to which the weighted coefficients are applied may be determined to be a fetus heart rate curve, which may need to be validated (block 824). The result of the validation step enables for the identification of a final or definitive and accurate fetus heart rate curve (block 828). In embodiments, as illustrated in FIG. 9, it may be determined, automatically and without user intervention or manually, whether the SP02 levels as determined in block 810 (e.g., oxygen levels present in the blood cells of the pregnant woman) are inversely correlated with the remnant signals to fetus heart rate curve, as determined in block 824. If an inverse correlation exists, it may be determined that the fetus heart rate curve determined in block 824 is potentially a final and accurate heart rate of the fetus. In embodiments, if there is a lack of an inverse correlation, additional physiological data, namely hemodynamic data may be collected and the steps described above and illustrated in FIG. 8-11, namely steps 802-824, may be repeated in order to identify the heart rate of the fetus.

Clause 1: A method of determining at least a physiological parameter specific to a fetus disposed in an individual, comprising: generating a spectral transform from a waveform representative of a hemodynamic parameter specific to the individual, the spectral transform including a plurality of harmonics within a sampling period, the plurality of harmonics representative of the hemodynamic parameter; determining a plurality of candidate remnant signals included in an area independent of exclusion areas of the spectral transform; identifying a candidate remnant signal from the plurality of candidate remnant signals; comparing the candidate remnant signal with a target signal representative of oxygen levels present in blood stream of the individual, the target signal is derived from the hemodynamic parameter; and determining that the candidate remnant signal is associated with a heart rate of the fetus that is disposed in the individual upon identifying an inverse correlation between the candidate remnant signal and the target signal of the individual.

Clause 2: The method of clause 1, further comprising obtaining, using a sensor of a device disposed on the individual, the hemodynamic parameter specific to the individual in whom the fetus is disposed.

Clause 3: The method of clause 1, wherein the hemodynamic parameter specific to the individual is the heart rate of the individual.

Clause 4: The method of clause 2, wherein the device is worn on a wrist of the individual.

Clause 5: The method of clause 2, wherein the device is disposed on a chest of the individual.

Clause 6: The method of any of clauses 1-5, further comprising performing one or more signal processing operations on the waveform representative of the hemodynamic parameter.

Clause 7: The method of any of clause 6, wherein the performing of the one or more signal processing operations include applying a low pass filter to the waveform representative of the hemodynamic parameter; and performing a detrending operation on the waveform representative of the hemodynamic parameter.

Clause 8: The method of any of clauses 1-7, wherein the identifying of the candidate remnant signal from the plurality of candidate remnant signals includes: merging at least two distinct candidate target remnant signals from the plurality of candidate remnant signals within an additional sampling period.

Clause 9: The method of clause 8, wherein the merging of the at least two distinct candidate target remnant signals includes: determining one or more weighted coefficients specific to the at least two distinct candidate target remnant signals; and applying the one or more weighted coefficients to the at least two distinct candidate target remnant signals for identifying the candidate remnant signal.

Clause 10: The method of clause 9, wherein the one or more weighted coefficients are weighted averages.

Clause 11: The method of any of clauses 1-10, further comprising determining that the candidate remnant signal is independent of the heart rate of the fetus upon identifying a correlation between the candidate remnant signal and the target signal of the individual.

Clause 12: The method of any of clauses 1-11, wherein the exclusion areas include the plurality of harmonics representative of the hemodynamic parameter of the individual.

Clause 13: The method of any of clauses 1-12, wherein the exclusion areas include a first set of frequencies below a threshold frequency value and a second set of frequencies above an additional threshold frequency value.

Clause 14: A device comprising: a sensor; at least one light source; a processor; a network communication interface; and memory; wherein the processor is configured to: generate a spectral transform from a waveform representative of a hemodynamic parameter specific to an individual, the spectral transform including a plurality of harmonics within a sampling period, the plurality of harmonics representative of the hemodynamic parameter; determine a plurality of candidate remnant signals included in an area independent of exclusion areas of the spectral transform; identify a candidate remnant signal from the plurality of candidate remnant signals; compare the candidate remnant signal with a target signal representative of oxygen levels present in blood stream of the individual, the target signal is derived from the hemodynamic parameter; and determine that the candidate remnant signal is associated with a heart rate of the fetus that is disposed in the individual upon identifying an inverse correlation between the candidate remnant signal and the target signal of the individual.

Clause 15: The device of clause 14, wherein the processor is further configured to obtain, using the sensor, the hemodynamic parameter specific to the individual in whom the fetus is disposed.

Clause 16: The device of any of clauses 14 or 15, wherein the hemodynamic parameter specific to the individual is the heart rate of the individual.

Clause 17: The device of any of clauses 14-16, wherein the device is worn on a wrist of the individual.

Clause 18: The device of any of clauses 14-17, wherein the device is disposed on a chest of the individual.

Clause 19: The device of any of clauses 14-18, wherein the processor is further configured to perform one or more signal processing operations on the waveform representative of the hemodynamic parameter.

Clause 20: The device of any of clauses 14-19, wherein the processor is configured to perform the one or more signal processing operations by: applying a low pass filter to the waveform representative of the hemodynamic parameter; and performing a detrending operation on the waveform representative of the hemodynamic parameter.

Clause 21: The device of any of clauses 14-20, wherein the processor is configured to identify the candidate remnant signal from the plurality of candidate remnant signals by: merging at least two distinct candidate target remnant signals from the plurality of candidate remnant signals within an additional sampling period.

Clause 22: The device of clause 21, wherein the processor is configured to merge the at least two distinct candidate target remnant signals by: determining one or more weighted coefficients specific to the at least two distinct candidate target remnant signals; and applying the one or more weighted coefficients to the at least two distinct candidate target remnant signals for identifying the candidate remnant signal.

Clause 23: The device of any of clauses 14-22, wherein the one or more weighted coefficients are weighted averages.

Clause 24: The device of any of clauses 14-23, wherein the processor is further configured to determine that the candidate remnant signal is independent of the heart rate of the fetus upon identifying a correlation between the candidate remnant signal and the target signal of the individual.

Clause 25: The device of any of clauses 14-24, wherein the exclusion areas include the plurality of harmonics representative of the hemodynamic parameter of the individual.

Clause 26: The device of any of clauses 14-25, wherein the exclusion areas include a first set of frequencies below a threshold frequency value and a second set of frequencies above an additional threshold frequency value.

Clause 27: The device of any of clauses 14-26, wherein the hemodynamic parameter is at least one of a heart rate of the individual, a pulse rate of the individual, or a blood pressure of the individual.

In the above description, an embodiment is an example or implementation of the invention. The various appearances of “one embodiment”, “an embodiment” or “some embodiments” do not necessarily all refer to the same embodiments.

Although various features of embodiments of the present invention may be described in the context of a single embodiment, the features may also be provided separately or in any suitable combination. Conversely, although embodiments of the present invention may be described herein in the context of separate embodiments for clarity, the invention may also be implemented in a single embodiment.

Embodiments of the invention may include features from different embodiments disclosed above, and embodiments may incorporate elements from other embodiments disclosed above. The disclosure of elements of some embodiments of the invention in the context of a specific embodiment is not to be taken as limiting their used in the specific embodiment alone.

Furthermore, it is to be understood that embodiments of the invention can be carried out or practiced in various ways and that embodiments of the invention can be implemented in other ways than the ones outlined in the description above.

The invention is not limited to the diagrams or to the corresponding descriptions contained herein. For example, in a method according to some embodiments of the present invention, the flow need not move through each illustrated step or state, or in exactly the same order as described.

Meanings of technical and scientific terms used herein are to be commonly understood as by one of ordinary skill in the art to which the invention belongs, unless otherwise defined.

While this specification refers to a limited number of embodiments, these should not be construed as limitations on the scope of the invention, but rather as exemplifications of some of the preferred embodiments. Other possible variations, modifications, and applications are also within the scope of embodiments of the present invention.

What is claimed is:

1. A method of determining at least a physiological parameter specific to a fetus disposed in an individual, comprising:
generating a spectral transform from a waveform representative of a hemodynamic parameter specific to the individual, the spectral transform including a plurality of harmonics within a sampling period;
determining a plurality of candidate remnant signals included in an area independent of exclusion areas of the spectral transform;
identifying a candidate remnant signal from the plurality of candidate remnant signals;
comparing the candidate remnant signal with a target signal representative of oxygen levels present in blood stream of the individual, the target signal is derived from the hemodynamic parameter; and
determining that the candidate remnant signal is associated with a heart rate of the fetus that is disposed in the individual responsive to identifying an inverse correlation between the candidate remnant signal and the target signal of the individual.

2. The method of claim 1, further comprising obtaining, using a sensor of a device disposed on the individual, the hemodynamic parameter specific to the individual in whom the fetus is disposed.

3. The method of claim 1 or claim 2, wherein the hemodynamic parameter specific to the individual is the heart rate of the individual.

4. The method of claim 2 or claim 3, wherein the device is worn on a wrist of the individual.

5. The method of claim 2 or claim 3, wherein the device is disposed on a chest of the individual.

6. The method of any of claims 1-5, further comprising performing one or more signal processing operations on the waveform representative of the hemodynamic parameter.

7. The method of claim 6, wherein the performing of the one or more signal processing operations include:
applying a low pass filter to the waveform representative of the hemodynamic parameter; and
performing a detrending operation on the waveform representative of the hemodynamic parameter.

8. The method of any of claims 1-7, wherein the identifying of the candidate remnant signal from the plurality of candidate remnant signals includes:
merging at least two distinct candidate target remnant signals from the plurality of candidate remnant signals within an additional sampling period.

9. The method of claim 8, wherein the merging of the at least two distinct candidate target remnant signals includes:
determining one or more weighted coefficients specific to the at least two distinct candidate target remnant signals; and
applying the one or more weighted coefficients to the at least two distinct candidate target remnant signals for identifying the candidate remnant signal.

10. The method of claim 9, wherein the one or more weighted coefficients are weighted averages.

11. The method of any of claims 1-10, further comprising determining that the candidate remnant signal is independent of the heart rate of the fetus upon identifying a correlation between the candidate remnant signal and the target signal of the individual.

12. The method of any of claims 1-11, wherein the exclusion areas include the plurality of harmonics representative of the hemodynamic parameter of the individual.

13. The method of any of claims 1-12, wherein the exclusion areas include a first set of frequencies below a threshold frequency value and a second set of frequencies above an additional threshold frequency value.

14. A device comprising:
a sensor;
at least one light source;
a processor;
a network communication interface; and
memory;
wherein the processor is configured to:
generate a spectral transform from a waveform representative of a hemodynamic parameter specific to an individual, the spectral transform including a plurality of harmonics within a sampling period, the plurality of harmonics representative of the hemodynamic parameter;
determine a plurality of candidate remnant signals included in an area independent of exclusion areas of the spectral transform;
identify a candidate remnant signal from the plurality of candidate remnant signals;
compare the candidate remnant signal with a target signal representative of oxygen levels present in blood stream of the individual, the target signal is derived from the hemodynamic parameter; and
determine that the candidate remnant signal is associated with a heart rate of a fetus that is disposed in the individual upon identifying an inverse correlation between the candidate remnant signal and the target signal of the individual.

15. The device of claim 14, wherein the processor is further configured to obtain, using the sensor, the hemodynamic parameter specific to the individual in whom the fetus is disposed.

16. The device of claim 14 or claim 15, wherein the hemodynamic parameter specific to the individual is the heart rate of the individual.

17. The device of any of claims 14-16, wherein the device is worn on a wrist of the individual.

18. The device of any of claims 14-17, wherein the device is disposed on a chest of the individual.

19. The device of any of claims 14-18, wherein the processor is further configured to perform one or more signal processing operations on the waveform representative of the hemodynamic parameter.

20. The device of claim 19, wherein the processor is configured to perform the one or more signal processing operations by:
applying a low pass filter to the waveform representative of the hemodynamic parameter; and
performing a detrending operation on the waveform representative of the hemodynamic parameter.

21. The device of any of claims 14-20, wherein the processor is configured to identify the candidate remnant signal from the plurality of candidate remnant signals by:
merging at least two distinct candidate target remnant signals from the plurality of candidate remnant signals within an additional sampling period.

22. The device of claim 21, wherein the processor is configured to merge the at least two distinct candidate target remnant signals by:
determining one or more weighted coefficients specific to the at least two distinct candidate target remnant signals; and
applying the one or more weighted coefficients to the at least two distinct candidate target remnant signals for identifying the candidate remnant signal.

23. The device of any of claims 14-22, wherein the one or more weighted coefficients are weighted averages.

24. The device of any of claims 14-23, wherein the processor is further configured to determine that the candidate remnant signal is independent of the heart rate of the fetus upon identifying a correlation between the candidate remnant signal and the target signal of the individual.

25. The device of any of claims 14-24, wherein the exclusion areas include the plurality of harmonics representative of the hemodynamic parameter of the individual.

26. The device of any of claims 14-25, wherein the exclusion areas include a first set of frequencies below a threshold frequency value and a second set of frequencies above an additional threshold frequency value.
